(19) United States
(12) Patent Application Publication
(10) Pub. No.: US 2012/0295337 A1
(43) Pub. Date: Nov. 22, 2012

(54) ALGAE CULTURE SYSTEM

(76) Inventor: Alfonso Navarro, Santiago (CL)

(21) Appl. No.: 13/473,872

(22) Filed: May 17, 2012

(30) Foreign Application Priority Data
May 17, 2011 (CL) CL 1145-2011

(51) Int. Cl. C12M 1/42; C12N 1/12

(52) U.S. Cl. 435/257.1; 435/292.1

(57) ABSTRACT

A microalgae culture system that provides greater control of a culture due to distribution and the shape of its components. The system allows for the possibility to incorporate gases into the medium, resulting in an increased culture yield and lower energy consumption per unit volume. The system generally includes a pool with a circular mantle, PVC parts and a removable lid.

### RELATED APPLICATION

The present application claims priority to Chilean Application No. CL 1145-2011 filed May 17, 2011, which is incorporated herein in its entirety by reference.

### SCOPE OF THE INVENTION

The invention relates to a microalgae culture system that delivers a greater control of the culture due to the distribution and shape of its components, and the possibility to incorporate gases into the medium, resulting in an increased culture yield and reduced energy consumption per volume unit.

### DESCRIPTION OF THE PRIOR ART

Microalgae have been used in aquaculture as a food supplement and in the production of chemical compounds (Raja et al., 2008 (full citation infra in References section)), and more recently they have been proposed as an energy source for fuel production, offering several advantages over traditional cultures, such as high photosynthetic efficiency, high lipid content, continuous production of biomass and fast growth (Moo-Younga and Chisti, 1994; Sanchez et al, 2003; Miao and Wu, 2006; (full citations infra in References section)), and also because they are a renewable source with low emissions of pollutants into the atmosphere (CO₂ and SO₂).

While the biological bases of the microalgae culture are widely developed on a small scale, they lack culturing capacity on a large scale to produce biomass at a low cost. For intensive production of microalgae, two culture systems are mainly used, open systems or Raceway ponds and closed systems or photobioreactors (PBRs). In open systems, cultures are exposed to the atmosphere in a type of channel of large dimensions and are constantly stirred by a paddlewheel. PBRs are highly productive culture systems that allow for a greater culture yield per area and volume unit, compared to open systems (Sanchez et al, 2003; Khan et al. 2009 (full citations infra in References section)). PBRs can be made of plastic, glass and transparent PVC, among other materials, and in different shapes, horizontal, vertical, circular, etc.

The high productivity of PBRs is associated with the control of all culture parameters and the aseptic conditions they provide, which ultimately translate into higher productivity per volume unit. The production figures of both systems reach very different numbers. According to Sánchez, in terms of volume productivity in Kg/m³, photobioreactors are fifteen times more effective than open systems and use half the area measured in hectares. In addition to these advantages, open ponds can be contaminated and difficult to control in terms of culture conditions. However, installation costs are minimal compared to those incurred with PBRs, which at the same time are more difficult to clean. These are the main reasons that so far most of the microalgae production has been made in open systems.

Regarding the disadvantages of current systems using open ponds, the only production systems currently in use are open pools, of the raceway or circular type. These systems use a mechanical stirrer, of the paddlewheel type, which is in contact with the water and undergoes corrosion and wear. The movement resulting from this type of propulsion tends to be a laminar flow, which means an incorrect turbulence for the algae nutrition.

These systems were not designed to incorporate gases such as air or pure CO₂ into the medium, and the only source of CO₂ is a passive transfer of gas from the air into the water through the exposed surface. This creates a limitation in the capture of CO₂, resulting in low growth rates.

Escalation of these systems to sizes larger than current ones (100 ha) is impractical due to low productivity per area unit and the enormous loss of water by evaporation. These systems also tend to be contaminated with chemical and biological agents, such as larvae, bacteria, microalgae competing and predating species, which have presented serious problems for existing plants, significantly reducing the plant factor.

Finally, the use of moving parts or engines near or in contact with water increases the probability of failure, especially when using seawater.

### SUMMARY OF THE INVENTION

The system of the invention is a closed microalgae culture system that provides a greater control of the culture and the possibility to incorporate gases into the medium, resulting in an increased culture yield and lower energy consumption per volume unit. The invention uses a cellular model for ease of escalation, allowing the independence of the units and an escalation ad infinitum. The model physically includes arrays of circular ponds with transparent lids, airlift water pumping systems connected to a loading/unloading system, provision of gases and an automatic control system.

The above summary of the invention is not intended to describe each illustrated embodiment or every implementation of the present invention. The figures and the detailed description that follow more particularly exemplify these embodiments.

### BRIEF DESCRIPTION OF THE DRAWINGS

The embodiments of the present invention may be more completely understood in consideration of the following detailed description of various embodiments in connection with the accompanying drawings, in which:

FIG. 1 is a plan view of the pond without the lid and its elements according to an embodiment of the invention;

FIG. 2 is a side sectional view of the pond without the lid and its elements of FIG. 1;

FIG. 3 is a plan view of the pond with its hexagonal transparent lid according to an embodiment of the invention;

FIG. 4 is a side view of the pond with its hexagonal transparent lid according to FIG. 3;

FIG. 5 is a graph of cells/ml. considering Examples 1, 2 and 3;

FIG. 6 is an isometric view of the installation; and

FIG. 7 depicts an installation and distribution with six groups or cells of three ponds each.

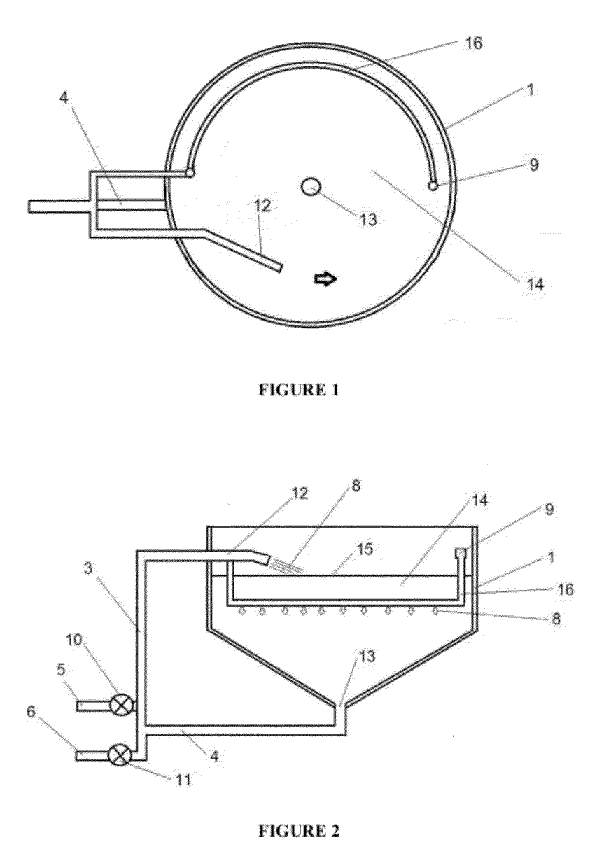

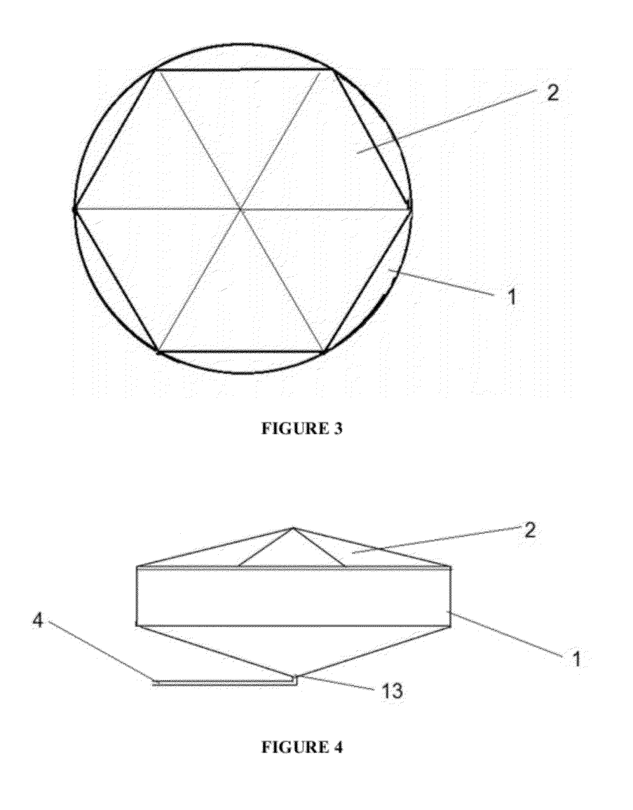

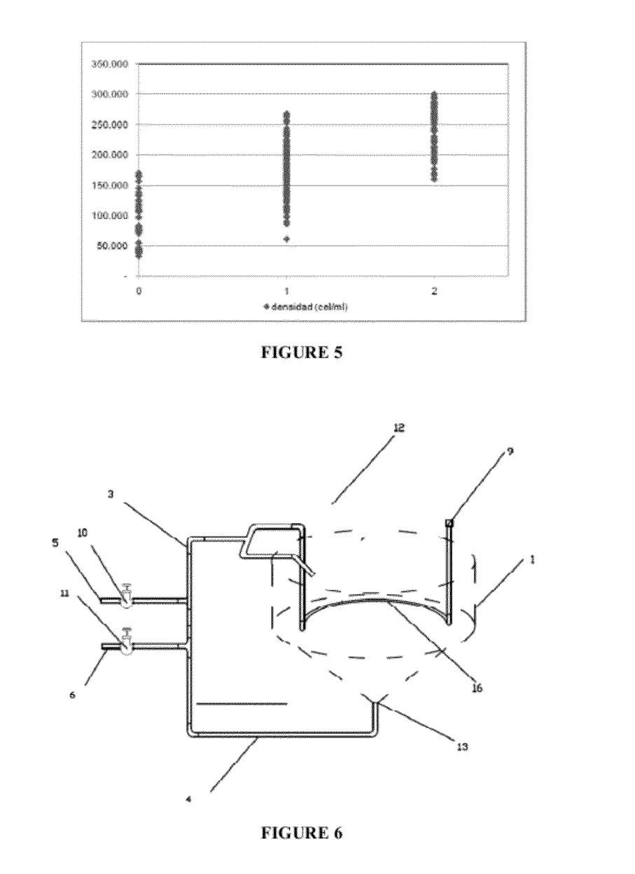

While the present invention is amenable to various modifications and alternative forms, specifics thereof have been shown by way of example in the drawings and will be described in detail. It should be understood, however, that the intention is not to limit the present invention to the particular embodiments described. On the contrary, the intention is to cover all modifications, equivalents, and alternatives falling within the spirit and scope of the present invention.

### DETAILED DESCRIPTION OF THE INVENTION

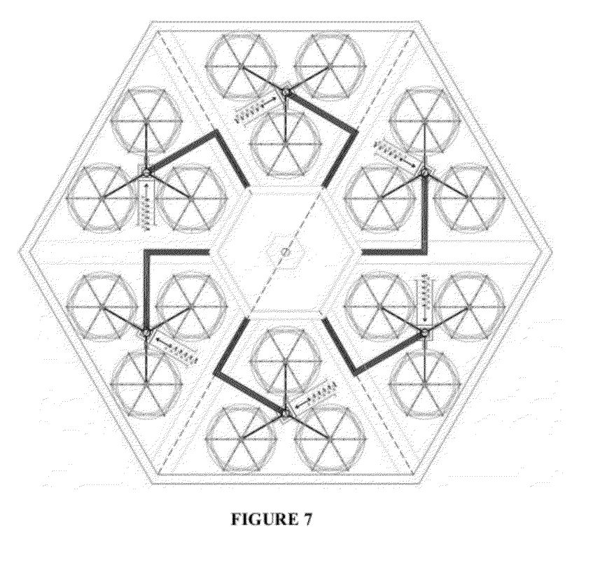

The culture system is designed considering escalation, that is, a spatial distribution of ponds that can easily increase the area of production by the incorporation of new cells (groups of three ponds connected to a central pumping system) (see FIG. 7).

Ponds are circular with a cylindrical mantle and conic bottom, preferably of fiberglass or coated cement. The preferred form of construction of the pond is that where the pond diameter is greater than its height.

During operation, ponds are covered by a hexagonal removable lid of a transparent material, preferably alveolar polycarbonate (FIGS. 3 and 4). For visual inspection of the pond interior, one of the faces of the hexagon can be removed.

The system keeps a stable temperature of the culture medium within a suitable range for the growth of microalgae. This is achieved because ponds are partially buried in the ground (FIG. 4, dotted line), which allows to counteract day-night thermal oscillations and not suffer significant variations in the average temperature between winter and summer. The ideal range of pH for the operation of the system is between 6.0 and 11.0.

The culture system performs propulsion by air. This propulsion system operates connected to a blower outside the installation (5), this blower allows to feed several modular groups or cells at the same time, producing a mild pumping while performing the gas exchange between the culture medium and the air pumped. The culture medium is distributed into the ponds in the movement of recirculation to maintain a constant stirring, to this end each pond has in its interior a first aerator (12) which carries the culture medium and the air, the first aerator (12) comprising, or alternatively consisting of, a straight tube near the liquid surface and above it at an angle between 30° and 60° respect to the liquid surface, which discharges into the pond tangentially against the cylindrical mantle of such pond, forming a circular flow or vortex which is also part of the propulsion system of the culture medium inside the pond.

The second PVC aerator carries the culture medium and the air, and comprises, or alternatively consist of, a semicircular tube (16) with a plug at one end (9), located near the bottom of such pond and which is part of the propulsion system of the culture medium inside the pond. Said second aerator comprises structure defining perforations through which the mixture of air or gas and culture medium is injected into the pond, such perforations are directed towards the bottom of the pond in order to prevent clogging of the holes by decantation of the cells in the culture (8).

Loading of the ponds with the culture medium and algae is made through the hydraulic line (6). Once the pond (1) is filled with culture medium (14), recirculation starts. Passage of pressurized gasses (5) is opened through the gas inlet valve (10), and these may be air, carbon dioxide or a mixture thereof, and the hydraulic line (11) is closed. The gases push the culture medium upwards and partially mix with water, which returns to the pond (1) entering tangentially against the circle. This movement generates a circular current. The culture medium is mixed and releases photosynthesis gases that mix with the pressurized gases at the top of the pond.

After spinning around the system, the culture medium returns to the center of the pond to the recirculation line (4). This line allows filling of the ponds with culture medium (14), incorporation of the inoculums, and harvesting is performed.

Air line (5) incorporates gases into the system (air) to supply power for the recirculation.

The system described in this application has no moving or metallic parts, which gives it a great versatility with respect to the photoreactors described in the state of the art. This feature makes it possible to use the invention for culturing microalgae either from fresh or salt water, among which are Arthrospira platensis, Monoraphidium graphitti, Chlorella vulgaris, Anabaena variabilis and Nannochloropsis oculata, Chlorella neustonica, respectively.

### EXAMPLES

### Example 1

Growth of the Arthrospira platensis microalgae in the photobioreactor described in this application:

Modified Zarrouk culture medium is used, in an agricultural degree;

Ambient air is used as a source of CO₂;

Density measurements were performed by cell counting using Sedgewick-Rafter chamber. The results are expressed in cells per milliliter.

Harvest was weighed completely dry and is expressed in grams. Performance calculations are made by adding the period harvesting, divided by the culture area (14 m²) and the number of days in the period, the results being expressed in grams per square meter per day (g/m²/day). Rest days and no-harvesting days are included.

### Example 2

Under standard culture conditions for Arthrospira platensis described in example 1, a continuous harvesting was conducted for 1 week. As a harvesting criterion, harvesting was performed each time the system exceeded an average of 180,000 cells per milliliter.

TABLE 1
Harvesting week 1

| | Harvest | Harvesting | Dry Weight | Harvest | Harvesting | |
|---|---|---|---|---|---|---|
| Date | Volume¹ | Time² | (Net)³ | Volume⁴ | %⁵ | Accumulated⁶ |
| Feb. 07, 2011 | 13 | 33 | 209 | 429 | 8% | 209.0 |
| Feb. 08, 2011 | 14.3 | 30 | 215 | 429 | 8% | 424.0 |
| Feb. 10, 2011 | 14.8 | 60 | 296 | 888 | 17% | 720.0 |
| Feb. 11, 2011 | 13 | 90 | 327 | 1170 | 22% | 1047.0 |

| | | | | | |
|---|---|---|---|---|---|
| Total time | 5 | days | Average Productivity | 15 | g/m²/day |
| Total | 1047.0 | g | Harvested Quantity | 56% | |
| Productivity | | | | | |

¹expressed in l/min;
²in minutes;
³in grams;
⁴1 * min;
⁵% of total volume 5.200 l;
⁶grams harvested.

### Example 2

Under standard culture conditions for Arthrospira platensis, a continuous harvesting was conducted for a 1 week. This time the secondary bubbler was incorporated, which aims to increase turbulence, improve incorporation of CO₂ into the system and degassing of the medium. As a harvesting criterion, harvesting was performed each time the system exceeded an average of 180,000 cells per milliliter. The following table shows the harvesting results.

TABLE 2
Harvesting week 2

| | Harvest | Harvesting | Dry Weight | Harvest | Harvesting | |
|---|---|---|---|---|---|---|
| Date | Volume¹ | Time² | (Net)³ | Volume⁴ | %⁵ | Accumulated⁶ |
| Mar. 01, 2011 | 21 | 30 | 214 | 630 | 12% | 214.0 |
| Mar. 02, 2011 | 13 | 30 | 184 | 390 | 7% | 398.0 |
| Mar. 03, 2011 | 10 | 60 | 344 | 600 | 12% | 742.0 |
| Mar. 04, 2011 | 12 | 60 | 279 | 720 | 13% | 1021.0 |
| Mar. 05, 2011 | 18 | 120 | 557 | 2160 | 41% | 1578.0 |

| | | | | | |
|---|---|---|---|---|---|
| Total time | 5 | days | Average Productivity | 22.5 | g/m²/day |
| Total | 1578.0 | g | Harvested Quantity | 86% | |
| Productivity | | | | | |

¹expressed in l/min;
²in minutes;
³in grams;
⁴1 * min;
⁵% of total volume 5.200 l;
⁶grams harvested.

The following table shows the difference in performance after incorporating the secondary bubbler.

TABLE 3
Comparison between periods without the tube and those with the tube.

| | | | Average | |
|---|---|---|---|---|
| | Average | | performance | |
| Time | cells/ml | Increase (%) | (g/m²/day) | Increase (%) |
| 0 | 89,280 | | | |
| 1 | 176,503 | | 5.1 | |
| 2 | 244,328 | 38% | 8.1 | 57% |

The foregoing descriptions present numerous specific details that provide a thorough understanding of various embodiments of the invention. It will be apparent to one skilled in the art that various embodiments, having been disclosed herein, may be practiced without some or all of these specific details. In other instances, components as are known to those of ordinary skill in the art have not been described in detail herein in order to avoid unnecessarily obscuring the present invention. It is to be understood that even though numerous characteristics and advantages of various embodiments are set forth in the foregoing description, together with details of the structure and function of various embodiments, this disclosure is illustrative only. Other embodiments may be constructed that nevertheless employ the principles and spirit of the present invention. Accordingly, this application is intended to cover any adaptations or variations of the invention.

For purposes of interpreting the claims for the present invention, it is expressly intended that the provisions of Section 112, sixth paragraph of 35 U.S.C. are not to be invoked unless the specific terms “means for” or “step for” are recited in a claim.

### REFERENCES

Murray Moo-Young and Yusuf Chisti (1994). Bioreactor applications in waste treatment. Resources, Conservation and Recycling. Vol 11, 13-24
Asterio Sánchez Miróna, M. Carmen Cerón García, Antonio Contreras Gómeza, Francisco García Camachoa, Emilio Molina Grimaa and Yusuf Chisti (2003). Shear stress tolerance and biochemical characterization of Phaeodactylum tricornutum in quasi steady-state continuous culture in outdoor photobioreactors. Biochemical Engineering Journal. Vol 16 (3), 287-297
Han Xua, Xiaoling Miao and Qingyu Wu (2006). High quality biodiesel production from a microalga Chlorella protothecoides by heterotrophic growth in fermenters. Journal of Biotechnology. Vol 126(4), 499-507
Shakeel A. Khan, Rashmib, Mir Z. Hussaina, S. Prasada and U. C. Banerjeeb (2009). Prospects of biodiesel production from microalgae in India. Renewable and Sustainable Energy Reviews, Vol 13 (9), 2361-2372.
Raja, R., Hemaiswarya, S., Kumar, N. A., Sridhar, S., & Rengasamy, R. (2008). A perspective on the biotechnological potential of microalgae. Critical Reviews in Microbiology, 34 (2), 77-88.

1. A closed microalgae culture system that provides a greater control of a microalgea culture and possibility to incorporate gases into a culture medium of the culture system, wherein the system uses a cellular model, the system comprising:
one or more groups of three bioreactor-type culture units, distributed so as to facilitate its escalation, each culture unit of such cellular model including
a pond with a cylindrical mantle and conic bottom buried in ground;
a hexagonal transparent lid installed on such pond;
a first aerator which carries the culture medium and air, the first aerator comprising a straight tube near a liquid surface and above it at an angle between 30° and 60° respect to the liquid surface, the first aerator being adapted to discharge into the pond tangentially against the cylindrical mantle of such pond, forming a circular flow or vortex thereby forming part of a propulsion system of the culture medium inside the pond;
a second aerator which carries the culture medium and the air, the second aerator comprising a semicircular tube with a plug at one end located near a bottom of such pond, the second aerator being part of the propulsion system of the culture medium inside the pond, said second aerator comprises perforations through which the mixture of air or gas and culture medium is injected into the pond, such perforations are directed towards the bottom of the pond;
a recirculation line from the bottom of the pond to the first and second aerators, along which an air injection line is inserted;
a pipe and a valve defining a gas inlet line; and
a pipe and a valve defining a liquid inlet line.

2. The microalgae culture system according to claim 1, wherein the injection of air into the system is done by using an external blowing system adapted to feed several cellular model units, such blower adapted to inject a pressure of about 0.1 bar.

3. The microalgae culture system according to claim 1, wherein the gas inlet line also allows direct injection of carbon dioxide.

4. The microalgae culture system according to claim 1, wherein a combination of flows from the first aerator and the second aerator is used to improve aeration.

5. The microalgae culture system according to claim 1, wherein a flow of the perforations of the second aerator oriented towards the bottom of the pond is also adapted to prevent decantation in such pond.

6. The microalgae culture system according to claim 1, wherein the pond is buried in the ground for improvements in thermal isolation and to keep a constant temperature of the culture medium.

7. The microalgae culture system according to claim 1, wherein inoculums are loaded directly into the pond.

8. The microalgae culture system according to claim 1, wherein a pH of the culture medium is between about 6.0 and about 11.0.

9. The microalgae culture system according to claim 1, wherein the system contains no metallic parts such that the system can be used either with fresh or salt water.

10. The microalgae culture system according to claim 1, wherein a pond diameter of the pond is greater than a height of the pond.

11. The microalgae culture system according to claim 1, wherein the hexagonal transparent lid is made of polycarbonate.

13. The microalgae culture system according to claim 1, wherein the second aerator is made from polyvinylchloride (PVC).

12. A method for using a culture system, wherein the method comprises:
providing a culture unit including
a pond with a cylindrical mantle and conic bottom;
a hexagonal transparent lid installed on such pond;
a first aerator adapted to carry a culture medium of the system and air, the first aerator comprising a straight tube near a liquid surface of the pond and above the pond at an angle between 30° and 60° respect to the liquid surface, the first aerator being adapted to discharge into the pond tangentially against the cylindrical mantle of such pond, thereby forming a circular flow or vortex thereby forming part of a propulsion system of the culture medium inside the pond working in combination with the first aerator;
a second aerator adapted to carry the culture medium and the air, the second aerator comprising a semicircular tube with a plug at one end located near a bottom of such pond and which is part of the propulsion system of the culture medium inside the pond, said second aerator comprises perforations through which the mixture of air or gas and culture medium is injected into the pond, such perforations being directed towards the bottom of the pond;
a recirculation line from the bottom of the pond to the first and second aerators, along which an air injection line is inserted;
a pipe and a valve defining a gas inlet;
a pipe and a valve defining a liquid inlet;
a pipe for liquid inlet, through which the pond is loaded with culture medium and microalgae;
filling the pond;
once the pond is filled, staring recirculation, and closing the liquid inlet;
opening the gas inlet valve to allow gases to push the culture medium upwards and partially mix with water, which returns to the pond entering through first and second aerators;
allowing for, after spinning around the system, return of the culture medium to a center of the pond to the recirculation line; and
once relevant measurements are performed, harvesting the medium using the recirculation line.

13. The method of claim 12, wherein the hexagonal transparent lid is made of polycarbonate.

14. The method of claim 12, wherein the second aerator is made of polyvinyl chloride (PVC).
